(19) United States
(12) Patent Application Publication
(10) Pub. No.: US 2026/0250461 A1
(43) Pub. Date: Aug. 27, 2026

(54) REUSABLE RESINS AND COMPOSITES

(71) Applicant: Polynt Composites USA Inc., Carpentersville, IL (US)

(72) Inventors: Owen H. Decker, Smithville, MO (US); Robert M. Adams, Overland Park, KS (US); Michael Allen Mathews, Glenview, IL (US)

(21) Appl. No.: 19/550,112

(22) Filed: Feb. 25, 2026

Related U.S. Application Data
(60) Provisional application No. 63/763,015, filed on Feb. 25, 2025.

(51) Int. Cl. C08G 69/40; C07D 407/12; C08K 7/04

(52) U.S. Cl. CPC C08G 69/40; C07D 407/12; C08K 7/04

(57) ABSTRACT

Polyfuran monomers having a structure of Formula I, II or III are combined with a polymaleimide monomer to form reusable resins. The reusable resins are used to form polymer articles and composite articles that also include fibrous reinforcing materials. The reusable resins are recoverable from a composite article or a polymer article and can be reused as subsequent resin-forming matrixes and composite articles or for subsequent polymer articles.

### CROSS-REFERENCE TO RELATED APPLICATIONS

This patent application claims priority to U.S. Provisional Patent Application No. 63/763,015, filed on Feb. 25, 2025, the entire contents of which are incorporated herein by reference.

### BACKGROUND

Recycling of products after their initial use can provide many benefits for the environment and the economy, from saving energy, water, and raw materials to decreasing waste in landfills and greenhouse gas emissions. Recycling can also save costs, provide jobs, expand existing markets, and create new opportunities.

Recycling of polymer composite materials is highly desirable, as millions of metric tons are used annually throughout the world. Polymer composite materials generally comprise a reinforcing material (such as carbon fiber) held together by a matrix (such as a thermoplastic or thermoset polymer). The matrix is generally formed by hardening and/or solidifying a resin applied to the reinforcing material. Use of composite materials continues to grow due to increasing demand from various sectors, such as construction, transportation, marine and aerospace. The demand is driven by the many advantages polymer composite materials enjoy, such as high strength to weight ratio, flexibility, and long product life, and to the ease of formulating composites with properties tailored for their intended use.

Many existing polymer composite materials can be recycled, though some are limited to “open-loop recycling,” in which a material is recovered but cannot be employed for its original use; but instead may be employed for other uses. Examples of open-loop recycling are the use of broken concrete as railroad ballast, and the use of cured, crushed solid epoxy resin as a filler. “Closed-loop recycling” refers to re-use of reclaimed material in an application like its original use without loss of its properties. Examples of closed-loop recycling used in other fields include making new steel articles by remelting of scrap steel, or re-smelting of scrap copper. In the context of polymer composite materials, a highly beneficial closed-loop recycling approach would entail the removal of a resin from a polymer composite after its use and then re-use of the resin in a new composite matrix.

Successful recycling of polymeric resins has generally been limited to thermoplastics. Thermoplastic polymers such as polyethylene, polypropylene and polyethylene terephthalate can be reheated and remolded into new articles, and this property provides an advantage with respect to recycling. Similarly, most practical closed-loop recycling of matrix resins has been of thermoplastic polymers. In the context of composite materials, thermoplastic resins are generally coupled with fibers that are cut short or ‘chopped’ so that the fiber/resin compositions may be processed in rotary extruders with intermeshing elements. When such compositions are recycled for re-use, the chopped fibers, pigments, fillers, or other solid amendments are not separated from the resin, but are carried with the resin into the re-use.

A set of products from Arkema, trade-named ELIUM, is advertised as being recyclable liquid thermoplastic resin for composites. Dubois US20220324140A1 discusses a process for recycling a first article to be recycled including a composite material based on a fibrous reinforcer and a thermoplastic, preferably a (meth)acrylic, polymer matrix. See also Dubois US20110160419A1 and Gerard US20160017138A1.

In recent years, there has been considerable work on a class of materials called “vitrimers.” Vitrimers have also been referred to as “covalent adaptable networks”. Vitrimers differ from traditional thermosetting resins in that the chemical reactions by which they crosslink are reversible at accessible temperatures. A commercially available class of vitrimer is a set of imines called VITRIMAX from Mallinda Corporation. Taynton et al. US20200247937A1 discusses covalent network polymers prepared from an imine-linked oligomer and an independent crosslinker comprising reactive moieties selected from the group consisting of epoxy, isocyanate, bismaleimide, sulfide, polyurethane, anhydride, polyester and combinations thereof. See also Taynton et al. US20220372273A1.

Gosau et al. US20190047181A1 relates in general to recycling of polymer matrix composite and discusses a process for separating reinforcement material from a thermoset polymer matrix.

Sommerfeldt et al. WO2024125740A1 discusses a method of extracting thermoset epoxy fractions from a composite structure comprising thermoset epoxy matrix having a network structure and embedded solid element(s).

There remains a need for a matrix-forming resin which can be recovered from polymer composite materials and re-used in closed-loop recycling, in the same form for the same or similar articles, especially in an efficient and/or environmentally-friendly manner.

### SUMMARY

As one aspect of the present disclosure, polyfuran monomers having at least two furan functionalities per molecule are provided. In some embodiments, the polyfuran monomers have a structure of Formula I, II or III. As another aspect, methods of making polyfuran monomers by reacting a polyisocyanate and a furfuryl alcohol are provided.

In another aspect of the present disclosure, reusable resins (e.g., a reusable matrix-forming resins) are provided. The reusable resins comprise one or more polyfuran monomers having at least two furan functionalities per molecule, and one or more polymaleimide monomers having at least two maleimide functionalities per molecule. As another aspect, composite compositions are provided which comprise the reusable matrix-forming resin and a fibrous reinforcing material. As yet another aspect, polymerizable compositions are provided which comprise the reusable resin.

The present disclosure also provides methods of recovering a reusable resin from a polymer article. In some embodiments, a reusable matrix-forming resin is recovered from a composite article that also comprises a fibrous reinforcing material. The reusable resin comprising one or more polyfuran monomers and one or more polymaleimide monomers can be reclaimed and reused for subsequent matrixes and composite articles or for subsequent polymer articles.

Other aspects of the present disclosure include methods of forming a composite article or a polymer article, and processes are provided for repairing or reforming a composite article made from the present reusable resins.

These and other features and advantages of the present methods and compositions will be apparent from the following detailed description, in conjunction with the appended claims.

### BRIEF DESCRIPTION OF THE DRAWINGS

FIG. 1 shows the measured viscosities of an embodiment of the present reusable matrix-forming resins before and after recovery from a composite article.

The present teachings are best understood from the following detailed description when read with the accompanying drawing FIGURES. The features are not necessarily drawn to scale. Wherever practical, like reference numerals refer to like features.

### DETAILED DESCRIPTION

The present disclosure provides a reusable resin (e.g., a reusable matrix-forming resin) which can be recycled after use in a composite article or a polymer article. The present disclosure also provides several methods of making and using such resins. Various embodiments may have one or more of the following advantages: the present resins can form a matrix or polymer by a chemical crosslinking reaction that is reversible in a practical procedure; the present resins are prepared from readily available starting materials, including bioavailable materials; the present resins, polymerizable compositions, composite compositions, and methods of making and using them are scalable to bulk manufacturing equipment; the present resins polymerizable compositions, and composite compositions are easily adjustable to provide control of composite mechanical performance; the polymers, matrix and composite articles made with the present resins are low in color; the polyfuran and polymaleimide monomers of the reusable resin are either liquid at convenient temperatures, or easily melted or dissolved; and/or the composite composition and reusable resins are usable in a variety of applications where fiber/resin composites are typically used.

The present reusable resin employs a Diels-Alder reaction to harden or solidify the resin and form the matrix. The Diels-Alder reaction is a reversible 4+2 cycloaddition of an electron-rich diene and an electron-deficient dienophile. An especially useful version is the Diels-Alder addition between a substituted furan and an N-substituted maleimide as illustrated below.

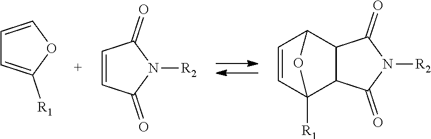

Diels-Alder reactions of furan and maleimide are reviewed in Gandini et al., “The furan/maleimide Diels-Alder reaction: A versatile click-unclick tool in macromolecular synthesis”, Progress in Polymer Science 38 (2013) 1-29.

As one aspect, reusable resins (e.g., a reusable matrix-forming resins), composition compositions, polymer compositions, and methods of preparing such compositions are provided. The resins and compositions comprise one or more polyfuran monomers and one or more polymaleimide monomers. Substantially all of the polymaleimide monomers have two or more maleimide functionalities per molecule, and substantially all of the polyfuran monomers have two or more furan functionalities per molecule: In some embodiments, the polyfuran monomer has at least an average of 2.25 furan functionalities per molecule, or at least an average of 2.5 furan functionalities per molecule. Functionality is determined by the structure of the compounds from which polyfuran monomers are made, and by their combining ratios. For example, when a mole of a diisocyanate (containing two moles of isocyanate functional groups) is reacted with two moles of furfuryl alcohol, the resulting polyfuran monomers will each have two furan functional groups, connected by urethane linkages from the isocyanates. If a mole of diisocyanate is instead reacted with a mixture of furfuryl alcohol and trimethylol propane, chosen to provide a total of two moles of alcohol functional groups, the resulting material will average more than two furan functional groups per molecule. Making the typical assumptions of complete reaction and absence of side reactions, the functionality, or average number of furan groups per molecule, may be determined by mathematical computation. The functionality of the polymaleimides may be similarly determined. For example, if one begins with 1,6-hexanediamine, and transforms the amino functional groups into maleimide functional groups, the purified product will contain two maleimide functional groups per molecule. In some embodiments, the furan and maleimide functionalities are present in the composition in a ratio from about 2 furan functionalities per maleimide functionality to about 0.5 furan functionalities per maleimide functionality.

### Polyfuran Monomers

The present compositions and methods employ polyfuran monomers which have two or more furan functionalities. In some embodiments, the polyfuran monomer has 3 or more, 4 or more, 5 or more, or 6 or more furan functionalities. In some embodiments, the polyfuran monomer has 2 to 10, or 2 to 8, or 3 to 10, or 3 to 8, or 3 to 6. As used herein, “furan functionality” and “furan functional group” are generally interchangeable and mean a 5-member heterocycle containing an oxygen atom and conjugated diene. A furan functional group can be unsubstituted (other than or in addition to its bond to the rest of the monomer) or have one or more substituents. The present methods generally produce mixtures of related polyfuran monomers having different numbers of functional groups. For example, a mixture having a computable average of 2.75 furan functional groups will contain monomers having 2, 3, 4, or 5, or more furan functional groups as statistically determined by molar ratios of the chemical compounds from which they are made. The presence of these higher molecular weight species can be corroborated by analytical techniques such as gel permeation chromatography.

In some embodiments, the polyfuran monomers comprise one or more compounds of formulas I, II and III shown below:

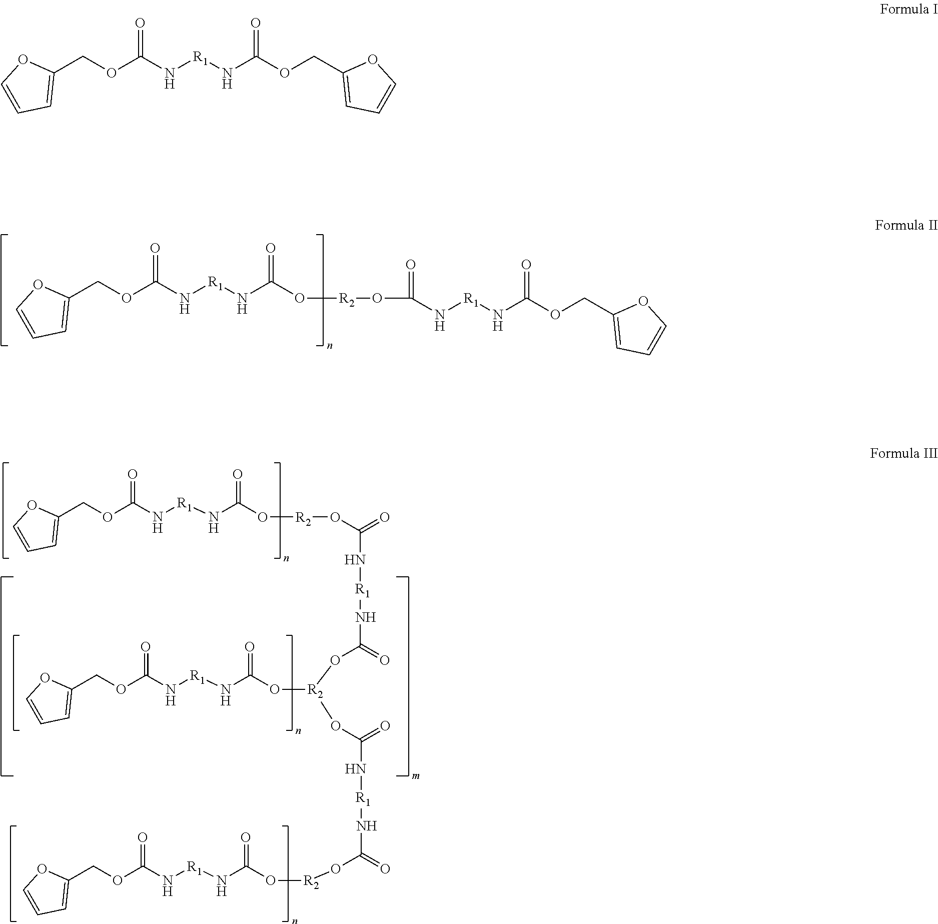

In Formulas I, II and III, R¹ is an aliphatic, cycloaliphatic, cycloaliphatic-aliphatic, aromatic, aromatic-aliphatic, heteroaromatic or heteroaromatic-aliphatic group, all of which are optionally substituted; R² is an aliphatic, cycloaliphatic, cycloaliphatic-aliphatic, aromatic, aromatic-aliphatic, heteroaromatic or heteroaromatic-aliphatic group, all of which are optionally substituted; m is 0 or more, alternatively 0 to 6, alternatively 0, 1, 2 or 3; n is 1 or more, alternatively 2, 3 or 4; and p is 1 or more, alternatively 1 or 2. In some embodiments, R¹ is an optionally substituted C₁ to C₃₀ aliphatic, aromatic or aromatic-aliphatic group, and/or R² is an optionally substituted C₃ to C₃₀ aliphatic aromatic, or aromatic-aliphatic group.

In some embodiments of Formula I, R¹ is a cycloaliphatic, cycloaliphatic-aliphatic, aromatic, aromatic-aliphatic, heteroaromatic or heteroaromatic-aliphatic group, all of which are optionally substituted.

In some embodiments, R¹ comprises a group of Formula IVa or IVb:

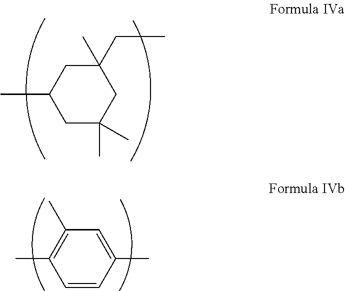

In some embodiments, R² is a group having a carbon framework selected to confer desired mechanical properties to polymers made from the matrix-forming resin. Such frameworks can be derived from diols or polyols. In some embodiments, the frameworks are derived from polyols having at least 3 hydroxy groups (for example, 3, 4, 5, 6 or more hydroxy groups), preferably carbon frameworks derived from polyols having primary alcohol groups, and more preferably from carbon frameworks derived from polyols having primary alcohol groups on non-adjacent carbons. Exemplary polyols include: (a) trimethylolethane, trimethylolpropane, pentaerythritol, di-trimethylolethane, di-trimethylolpropane, di-pentaerythritol, tripentaerythritol, 1,2,3,6-hexatetrol, 1,2,4-butanetriol, 1,2,5-pentanetriol, 2-methyl-propanetriol, 2-methyl-1,2,4-butanetriol, 1,3,5-trihydroxyethyl benzene, sugar alcohols such as pentoses and hexoses, dehydrated sugar alcohols such as sorbitan, (b) polyhydroxy functional esters of the polyols from group (a) with difunctional carboxylic acids, for example the bis(trimethylolpropane)ester of adipic acid, (c) polyethoxylated versions of the polyols from group (a), for example tris-ethoxylated trimethylolpropane. Examples of diols include 1,2-ethanediol, 1,2-propanediol, 1,3-propanediol, diethylene glycol, dipropylene glycol, 1,3-butanediol, 1.4-butanediol, 1,3-hexanediol, neopentyl glycol, 2-methyl-1,3-pentanediol, 1,3-butylene glycol, 1,6-hexanediol, hydrogenated bisphenol A, cyclohexane dimethanol, and 1,4-cyclohexanol.

In some embodiments, R² is a group having Formula V(a) to V(h):

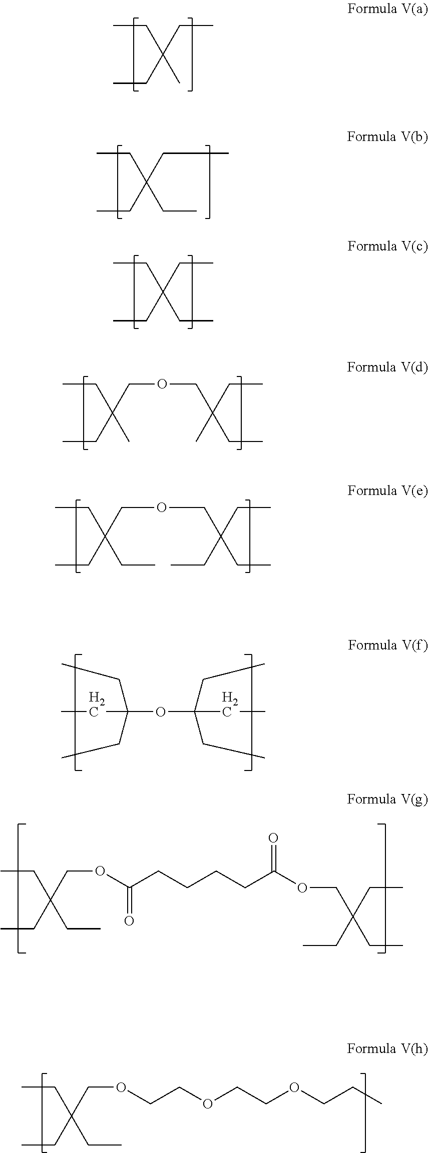

### Methods of Making Polyfuran Monomer

As another aspect of the present disclosure, methods are provided for making a polyfuran monomer. The methods comprise combining a furfuryl alcohol and a diisocyanate. Furfural (C₄H₃OCHO) can be used for production of the furfuryl alcohol. When furfuryl alcohol polymerizes in the presence of acids, it has a tendency to form dark-colored solid resins. This effectively limits the types of synthetic processes that may be used to create light-colored resins containing furan functional groups. For example, the acid-catalyzed direct esterification of carboxylic acids and alcohols that is so useful for assembling a plethora of esters and polyesters invariably produces dark materials when attempted with furfuryl alcohols. These dark-colored polymers are impractical to remove from materials that cannot be distilled. In some embodiments, polyfuran monomers are prepared without acids, such as by the addition of alcohols to isocyanates.

In some embodiments, a furfuryl alcohol and a diisocyanate are combined with one or more diols or polyols to form the polyfuran monomer. Examples of diols and polyols include those listed above. For instance, polyols include glycerol, trimethylolpropane, triethanolamine, tris-polyethoxylated trimethylolpropane, di-trimethylolpropane, pentaerythritol, di-pentaerythritol xylitol, sorbitol, and other carbohydrates. In some embodiments, the polyols have primary, non-adjacent hydroxy groups. For example, higher crosslink density provided by pentaerythritol or di-pentaerythritol can provide harder copolymers, whereas looser structure provided by a polyethoxylated trimethylol propane can provide tougher, more flexible copolymers.

Polyisocyanates employed in the present methods and compositions can be aromatic, aliphatic or cycloaliphatic polyisocyanates having two or more isocyanate groups per molecule. Polyisocyanates include but are not limited to diisocyanates and include aliphatic, cycloaliphatic, araliphatic, aromatic and heterocyclic polyisocyanates. Polyisocyanates include compounds of Formula VI:

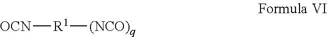

wherein q is equal to 1 to 3 and R¹ is as defined above for Formulas I, II and III. Exemplary diisocyanates include, but are not limited to, 1,4-Diisocyanatobutane (also referred to as 1,4-tetramethylene diisocyanate); 3-isocyanatomethyl-3,5,5-trimethylcyclohexyl isocyanate (also referred to as isophorone diisocyanate (IPDI); 2,4-diisocyanato-1-methylbenzene (also referred to as 2,4-toluene diisocyanate); 2,5-diisocyanato-1-methylbenzene; 2,6-diisocyanato-1-methylbenzene; 1,6-Hexane diisocyanate; 1,12-Dodecane diisocyanate; 1,3-Cyclobutanediisocyanate; 1,3-Cyclohexanediisocyanate; 1,4-Cyclohexanediisocyanate; 1-Isocyanato-3,3,5-trimethyl-5-(isocyanatomethyl)cyclohexane; 2,4-Diisocyanato-1-methylcyclohexane; 2,6-Diisocyanato-1-methylcyclohexane; 2,6-Diisocyanatocyclohexanone; Perhydro-4,4′-diphenylmethane diisocyanate; 1,3-Phenylenediisocyanate; 1,4-Phenylenediisocyanate; 4,4′-Diisocyanato-3,3′-dimethylbiphenyl; 4,4′-Diisocyanato-1,1′-biphenyl; 1,5-Naphthalene diisocyanate; 1,3-Xylylene diisocyanate; 1,4-Xylylene diisocyanate; 4,4′-Methylenebis(cyclohexyl isocyanate); 4,4′-Isopropylidenebis(cyclohexyl isocyanate); 1,4-Cyclohexanediisocyanate; 1-Isocyanato-3,3,5-trimethyl-5-(isocyanatomethyl)cyclohexane; 1-Methoxy-2,4-phenylenediisocyanate; 4-Chloro-1,3-phenylenediisocyanate; 4-(1-Isocyanatoethyl)phenyl isocyanate; 3-(3-Isocyanatobutyl)phenyl isocyanate; and 4-(2-Isocyanatocyclohexylmethyl)phenyl isocyanate. Mixtures of any of the above may be employed. In some embodiments, the diisocyanate can comprise one or more other substituents.

In some embodiments, the polyisocyanate is selected from 3-isocyanatomethyl-3,5,5-trimethylcyclohexyl isocyanate (also referred to as isophorone diisocyanate (IPDI); 2,4-diisocyanato-1-methylbenzene (also referred to as 2,4-toluene diisocyanate); 2,5-diisocyanato-1-methylbenzene; or 2,6-diisocyanato-1-methylbenzene. For polyisocyanates having more than one isomer, the present disclosure contemplates that a mixture of the isomers may be employed, or one or the other isomer can be selected based on the desired properties for the polyfuran monomer and/or the reusable matrix-forming resin. For example, IPDI has cis- and trans-stereoisomers, and the present methods and compositions can employ cis-IPDI, or trans-IPDI, or a mixture of cis-IPDI and trans-IPDI. The mixture can be a 50/50 mixture of cis- and trans-cis- and trans-isomers or another ratio. The two isocyanates of IPDI have different reactivities. This is true both for the cis-IPDI and the trans-IPDI. With proper choice of catalyst and temperature the secondary isocyanate on the cyclohexyl ring of both the cis-IPDI and the trans-IPDI is more than ten-fold more reactive than the isocyanate on the pendent methyl group. This difference in reactivity allows production of polyfuran resins of relatively low viscosity despite their high functionality. As another example, toluene diisocyanate (or diisocyanato-1-methylbenzene) is available as 2,4-, 2,5- and 2,6-isomers, and the present methods and compositions can employ one of the isomers or a mixture of those isomers. The structurally different isocyanates of diisocyanato-1-methylbenzenes, like those of IPDI, also differ in reactivity, although to a lesser degree than the two isocyanates in IPDI.

### Catalyst

In some embodiments, the methods of making a polyfuran monomer comprise combining the polyisocyanate and the furfuryl alcohol in the presence of an organometallic catalyst, for example, an organometallic catalyst containing tin, zinc, or bismuth. In some embodiments, the organometallic catalyst is tin dialkylalkanoates, tin dialkylmercaptides, tin dialkylbis (alkylmercaptoacetates), tin dialkylthioglycolates, zinc alkanoates, bismuth alkanoates, and mixtures thereof. In some embodiments, the alkyl groups and alkanoate groups of the organometallic catalyst can each have 1-20 carbon atoms, alternatively 4-12 carbon atoms, such as methyl, butyl, octyl or dodecyl (lauryl) groups. In some embodiments, the organometallic catalyst is selected from dialkyltin alkanoates, dialkyl tin mercaptides, dialkyl tin bis(alkylmercaptoacetates), dialkyltin thioglycolates and mixtures thereof. In some embodiments, the organometallic catalyst is a dialkyltin alkanoate such as dibutyltin dioctanoate or dibutyltin dilaurate.

### Polymaleimide Monomers

The present compositions and methods employ polymaleimide monomers which have two or more maleimide functionalities. As used herein, “maleimide functionality” and “maleimide functional group” are generally interchangeable and refer to a cyclic imide having an alkenyl group. A maleimide functional group can be unsubstituted (other than the bond connecting it to the rest of the polymaleimide monomer) or have one or more substituents.

Polymaleimide monomers include dimaleimide monomers which have two maleimide functional groups on average, including bis-maleimide monomers which have two identical maleimide functional groups attached to a central chemical moiety.

In some embodiments, the polymaleimide monomer is a compound of Formula VII:

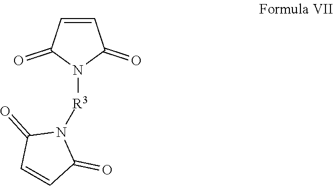

In Formula VII, R³ is aliphatic, cycloaliphatic, cycloaliphatic-aliphatic, aromatic, aromatic-aliphatic, heteroaromatic or heteroaromatic-aliphatic group, all of which are optionally substituted; Examples of R³ groups include, but are not limited to: —(CH₂)₁₋₈— (e.g., —CH₂—, —CH₂CH₂—, etc.); —CH₂CH₂C₆F₁₂CH₂CH₂—; —C₆H₄—; —C₆H₄—O—C₆H₄—; —C₆H₄—CH₂—C₆H₄—; —C₆H₄—O—C₆H₄—O—C₆H₄—; —C₆H₄—O—C₆H₄—C(CH₃)₂-C₆H₄—O—C₆H₄—; —C₆H₄—O—C₆H₄—C(CF₃)₂—C₆H₄—O—C₆H₄—; —C₆H₃CF₃—O—C₆H₄—C(CF₃)₂—C₆H₄—O—C₆H₃CF₃—; —C₆H₄—O—C₆H₄—(C═O)—C₆H₄—O—C₆H₄—; —C₆H₄—S—C₆H₄—C(CH₃)₂—C₆H₄—S—C₆H₄—; —C₆H₄—S(═O)₂—C₆H₄—; -4-methyl-1,3-phenylene bismaleimide; -3,3′-dimethyl-5,5′-diethyl-4,4′-diphenylmethane bismaleimide; —O—; and —O—C₆H₄—C(CH₃)₂—C₆H₄—O—.

In some embodiments, the polymaleimide monomer is selected from 1,6-hexamethylenebismaleimide, 4,4′-bismaleimidodiphenylmethane, and mixtures thereof.

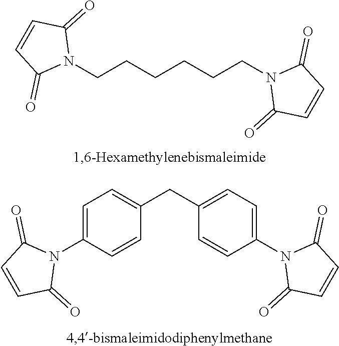 [SMILES: O=C1C=CC(=O)N1CCCCCCN1C(=O)C=CC1=O ; O=C1C=CC(=O)N1c1ccc(Cc2ccc(N3C(=O)C=CC3=O)cc2)cc1]

### Combined Method of Making Monomer and Resin

Another advantage of the present methods of preparing polyfuran/polymaleimide copolymers described above is that by forming the polyfuran monomer A, B, or C in a vessel, then adding the polymaleimide to form the copolymer D, E or F, it can be performed as a ‘one-pot’ process that does not resort to the use of solvents, filtrations or distillations. In some embodiments, the methods can begin with furfuryl alcohol and include the preparation of the polyfuran monomer in the same vessel. This feature makes the process especially useful for practical commercial manufacture of the polyfuran polymers and the polyfuran/polymaleimide copolymers in bulk.

### Composite Compositions and Composite Articles

The present reusable matrix-forming resins can be used with a reinforcing material such as glass fibers to form composite compositions which can be hardened to form composite articles. Generally composite articles are constructed of components which combine to produce structural or functional properties not present in any the separate components, such as added strength, efficiency, or durability. Composites generally comprise a reinforcing material held together by a matrix (such as a thermoplastic or thermoset polymer). Using a reinforcing material provides a way to strengthen or stiffen a polymerized resin and to form a variety of composite articles having many potential uses. After such uses of the composite, the matrix-forming resins can be reclaimed or recovered from the composite and used again in close-loop recycling.

In some embodiments, the reinforcing material are fibrous reinforcing material. Examples of fibrous reinforcing materials include glass fibers, quartz fibers, silica fibers, polyethylene fibers, carbon fibers, metal fibers, ceramic fibers, and others. For instance, the fibrous reinforcing material can be fibrous ceramics, e.g., alumina-silica (refractory ceramic fibers); boron fibers, silicon carbide, silicon carbide whiskers or monofilament, metal oxide fibers, including alumina-boric-silica, alumina-chromia-silica, zirconia-silica, and others. Organic polymer fibers include carbon fiber, fibrous graphite, acetates, acrylics (including acrylonitrile), aliphatic polyamides (e.g. nylon), aromatic polyamides, olefins (e.g., polypropylenes, polyesters, ultrahigh molecular weight polyethylenes), polyurethanes (e.g., Spandex), alpha-cellulose, cellulose, regenerated cellulose (e.g., rayon), jutes, sisal, vinyl chlorides, vinylidenes, flax, and thermoplastic fibers. Metal fibers include aluminum, boron, bronze, chromium, nickel, stainless steel, titanium or their alloys. The fibrous reinforcing material can also be single inorganic crystals.

The fibrous reinforcing material can be added in amount between 0 to 80% by weight, alternatively in an amount of 20 to 60% by weight based on the resin composition.

Composite articles include building materials, vehicle parts such as automobile panels and structures, marine parts such as boat hulls, aircraft parts such as wings, fuselages and control surfaces, sports equipment, enclosures or containers, wind turbine nacelles and spinners.

The composite articles, the materials for their production and processes may be used in addition to or in substitution for existing composite articles.

Annealing refers to any process that involves heating and cooling plastic materials to relieve stresses and/or improve stability. It is often used on thermoplastics, but it is not typically used on thermoset materials. However, the present inventors have found that composite articles formed with the present matrix-forming resins can be improved by annealing as described herein. Annealing can be performed at an annealing temperature or over an annealing range of temperatures over an annealing period. Annealing can be performed in a suitable temperature-controlled environment, such as a hot-air circulating drying furnace.

### Manufacture of Composite Articles

As another aspect of the present invention, methods are provided for manufacturing composite articles with the reusable matrix-forming resin. Composite articles can be manufactured using various procedures such as hand layup, pre-impregnated tape layup, liquid resin infusion; filament winding, spray molding, rotary molding, centrifugal molding, resin transfer molding, sheet molding, bulk molding, compression molding, or pultrusion. In a layup procedure, a composite article is formed by applying reinforcing material and matrix-forming resin in one or more layers each into an open mold.

In a liquid resin infusion procedure, the reinforcing material is applied and enclosed in an air-tight environment, such as a vacuum bag, against the surface of a mold or tool. An unpolymerized resin is then drawn into the reinforcement by means of applying a vacuum (i.e., a pressure lower than atmospheric pressure) across the flow length of the reinforcing material. Resin infusion of a typical hand lay-up process for making composites may improved by vacuum processing. Once layers of reinforcement and resin are laid-up by hand, the laminate is sealed in a vacuum bag and connected to a vacuum source. Vacuum is applied, creating mechanical pressure on the laminate throughout the cure cycle.

### Other Composition Components

In some embodiments, the present compositions comprise one or more composition components such as non-fibrous fillers. Examples of non-fibrous fillers are inert, particulate additives which can reduce the cost but also potentially alter some of the physical properties of the composite article. Accordingly the choice of fillers should be made with consideration of its overall effect on the desired properties. Examples of fillers include calcium carbonates, silicas, silicates, silicon dioxides, clays, feldspar, kaolin, flax, zirconia, calcium sulfates, micas, talcs, wood in various forms, glass (milled, platelets, spheres, micro-balloons), plastics (milled, platelets, spheres, micro-balloons), recycled thermoplastic composite particles, metals in various forms, metallic oxides or hydroxides, metal hydrides or metal hydrates, carbon particles or granules, alumina, alumina powder, aramid, bronze, carbon black, carbon fiber, cellulose, alpha cellulose, coal (powder), cotton, fibrous glass, graphite, jute, molybdenum, nylon, orlon, rayon, silica amorphous, sisal fibers, fluorocarbons and wood flour.

In some embodiments, the present matrix-forming compositions comprise one or more composition components such as pigments, thinners, wetting agents, surfactants, flow promoting agents, leveling agents, degassing agents, antifoam agents, texturizing agents, thickeners, rheology modifiers, antioxidants, heat stabilizers, ultraviolet light stabilizers, free-radical scavengers, color-stabilizing agents, antimicrobial agents, preservatives, bacteriocides, or fungicides.

### Method of Recovering Reusable Resins

As another aspect of the present disclosure, methods are provided for recovering a reusable resin from a polymer article comprising a polyfuran/polymaleimide copolymer, for example, recovering a reusable matrix-forming resin from a composite article. The methods comprise contacting the polymer article or composite article with a polar aprotic solvent at a high temperature, such as such as 150° C. or higher and dissolving the copolymer. The resin may dissolve without alteration of its molecular architecture. Alternatively, at least some portion of the polyfuran/polymaleimide copolymer may undergo reversal of the Diels-Alder reaction, reconstituting furan functionalities and maleimide functionalities, which may increase the solubility of oligomeric species in the solvent.

The methods comprise precipitating the dissolved copolymer, and separating the solvent from the precipitated copolymer. In some embodiments, solvent residues are removed from the separated copolymer. The solvent can be removed by using a vacuum or a reduced pressure to drive off a solvent from a mixture or another technique.

The present methods of reclaiming the present reusable resin (e.g., a reusable matrix-forming resin) can employ a solvent having a high boiling point, such as 150° C. or higher. Examples of high-boiling point solvents include ketone solvents such as 2-heptanone, 3-heptanone, 4-heptanone, cyclohexanone, 4-hydroxy-4-methyl-2-pentanone, and 3,5,5-trimethylcyclohex-2-en-1-one. Other examples of high-boiling point solvents include mesitylene (1,3,5-trimethylbenzene), anisole (methoxybenzene), pseudocumene (1,2,4-trimethylbenzene), propylbenzene, cumene (propan-2-yl)benzene, and n-butylbenzene. In some embodiments, the solvent is a mixture of high-boiling point ketone and aromatic solvents, such as a mixture of 2-heptanone and mesitylene.

In some embodiments, the solvent is a high-boiling point, polar aprotic solvent. Examples of high-boiling point, polar aprotic solvents include dimethylformamide (DMF), dimethylacetamide (DMAC), dimethyl sulfoxide, sulfolane, dimethylsulfolane, N,N′-dimethyl propyleneurea (DMPU), hexamethylphosphoramide (HMPA), dipropylene glycol dimethyl ether (DMM), bis(2-methoxyethyl)ether, benzonitrile, o-tolunitrile, p-tolunitrile, and mixtures thereof. In some embodiments, the present methods employ a solvent having a lower boiling point when the methods further comprise applying an elevated pressure to the solvent in which the composite article is placed. The elevated pressure should be sufficiently high to avoid significant evaporation of the solvent. Examples of such solvents include acetone, acetonitrile, dioxolane, dioxane, diethyl ether, diisopropylethylamine, dimethoxyethane (DME), ethylbenzene, methyl tert-butyl ether (MTBE), N-methylmorpholine, N-methylpyrrolidine, N-methylpiperidine, propionitrile, tetrahydrofuran (THF), triethylamine, toluene, xylene, and mixtures thereof.

### Polymer Articles

In some embodiments, the reusable resin is used without a reinforcing material as a polymerizable curable composition and/or to form a polymer article. For example, the reusable resin can be used a sealant, an adhesive, a coating, a laminating material, or a casting material. Methods of forming a polymer article from the present reusable resin can comprise injection molding, compression molding, blow molding, rotational molding, resin casting, vacuum casting, and additive manufacturing or 3D printing.

### Terminology

It is to be understood that the terminology used herein is for the purpose of describing particular embodiments only, and is not intended to be limiting, since the scope of the present teachings will be limited only by the appended claims. Unless defined otherwise, all technical and scientific terms used herein have the same meaning as commonly understood by those working in the fields to which this disclosure pertain. The defined terms are in addition to the technical and scientific meanings of the defined terms as commonly understood and accepted in the technical field of the present teachings.

“Resin” as used herein means any natural or synthetic monomer(s), oligomer(s), prepolymer(s), and combinations thereof which are adapted or intended for a reaction that results in a polymer. A “reusable resin” as used herein refers to a resin that can be removed, separated or otherwise recovered from a matrix, such as the matrix of polymer composite article, and re-used to form a matrix for a subsequent article. A reusable resin can be advantageously used in closed-loop recycling of composite articles. A reusable matrix-forming resin refers to a resin capable of or adapted for forming a polymerized solid phase capable of or adapted for holding reinforcing material in place.

“Monomer” as used herein means a molecule that is capable of or adapted for reacting with other molecules, which may be the same or different, to form a larger molecule such as a polymer. It should not be construed as being limited to a particular number of functional groups or a particular weight or size unless the context indicates otherwise.

“Matrix” refers to a polymerized solid phase capable of or adapted for holding reinforcing material in place. The matrix can be a continuous or partially continuous solid phase in which a reinforcing material is surrounded, embedded, or otherwise held together.

As used in the specification and the appended claims and in addition to its ordinary meaning, the terms “approximately” and “about” mean to within an acceptable limit or amount to one having ordinary skill in the art. The term “about” generally refers to plus or minus 15% of the indicated number. For example, “about 10” may indicate a range of 8.5 to 11.5. For example, “approximately the same” means that one of ordinary skill in the art considers the items being compared to be the same. It should be understood that any of the values disclosed herein are also a disclosure of the approximate value (e.g., the disclosure of “0.10” shall also constitute a disclosure of “about 0.10”), and any disclosure of an approximate value is a disclosure of the value itself (e.g., the disclosure of “about 0.10” shall also constitute a disclosure of “0.10”), unless the context indicates otherwise.

As disclosed herein, numeric ranges are provided for various parameters or data. It should be understood that numeric ranges also disclose include each intervening value within the range, to the tenth of the unit of the lower limit, unless the context clearly dictates otherwise. It should also be understood that the upper and lower limits may each be included or excluded in a range.

It should be recognized that chemical structures and formula may be elongated or enlarged for illustrative purposes.

The term “aliphatic” refers to hydrocarbon compounds which can be saturated or unsaturated and includes alkyl, alkenyl, and alkynyl compounds which can be substituted or unsubstituted. The term “alkyl” refers to a straight-chain or branched alkyl, preferably having 1, 2, 3, 4, 5, 6, 7, 8, 9, 10, 11, 12, 13, 14, 15, 16, 17, 18, 19, 20, 21, 22, 23 or 24 carbons. Examples of such alkyl groups include methyl, ethyl, propyl, isopropyl, n-butyl, sec-butyl, isobutyl, tert-butyl, pentyl, isoamyl, hexyl, and the like. The term “alkenyl” refers to a straight or branched hydrocarbon, preferably having 2, 3, 4, 5, 6, 7, 8, 9, 10, 11, 12, 13, 14, 15, 16, 17, 18, 19, 20, 21, 22, 23 or 24 carbons, and having one or more carbon-carbon double bonds. Nonlimiting examples of alkenyl groups include ethenyl, 1-propenyl, 2-propenyl (allyl), iso-propenyl, 2-methyl-1-propenyl, 1-butenyl, and 2-butenyl. The term “alkynyl” refers to a straight or branched hydrocarbon, preferably having 2, 3, 4, 5, 6, 7, 8, 9, 10, 11, 12, 13, 14, 15, 16, 17, 18, 19, 20, 21, 22, 23 or 24 carbons, and having one or more carbon-carbon triple bonds. Alkynyl groups include, but are not limited to, ethynyl, propynyl, and butynyl. Aliphatic groups may be unsubstituted or substituted by one or more suitable substituents.

“Heteroaliphatic” refers to an aliphatic group in which one or more carbon atoms have been replaced with a heteroatom, such as, O, N, or S. Any carbons within the aliphatic group can be replaced independently with a heteroatom (O, N, or S), meaning the first carbon, the terminal carbon or an internal carbon. For example, if the carbon atom of an alkyl group which is attached to the parent molecule is replaced with a heteroatom (e.g., O, N, or S) the resulting heteroalkyl groups are, respectively, an alkoxy group (e.g., —OCH₃, etc.), an amine alkyl (e.g., —NHCH₃, —N(CH₃)₂, etc.), or a thioalkyl group (e.g., —SCH₃). If a non-terminal carbon atom of the alkyl group which is not attached to the parent molecule is replaced with a heteroatom (e.g., O, N, or S) the resulting heteroalkyl groups are, respectively, an alkyl ether (e.g., —CH₂CH₂—O—CH₃, etc.), an alkyl amine (e.g., —CH₂NHCH₃, —CH₂N(CH₃)₂, etc.), or a thioalkyl ether (e.g., —CH₂—S—CH₃). If a terminal carbon atom of the alkyl group is replaced with a heteroatom (e.g., O, N, or S), the resulting heteroalkyl groups are, respectively, a hydroxyalkyl group (e.g., —CH₂CH₂—OH), an aminoalkyl group (e.g., —CH₂NH₂), or an alkyl thiol group (e.g., —CH₂CH₂—SH). A heteroaliphatic group can have, for example, 1 to 24 carbon atoms. A C₁-C₆ heteroalkyl group means a heteroalkyl group having 1 to 6 carbon atoms. A “substituted heteroaliphatic” means a heteroaliphatic as defined herein in which one or more hydrogen atoms has been replaced with a non-hydrogen substituent as defined in the “substituted” definition below.

The term “cycloaliphatic” refers to a monocyclic, bicyclic, or tricyclic moiety, preferably having 3, 4, 5, 6, 7, 8, 9, 10, 11, 12, 13, 14, 15, 16, 17, 18, 19, 20, 21, 22, 23 or 24 carbons, such as cyclobutanyl, cyclopentanyl, cyclohexyl, and cycloheptanyl. Cycloheteroaliphatic groups may be unsubstituted or substituted by one or more suitable substituents.

The term “cycloheteroaliphatic” refers to a monocyclic, bicyclic, or tricyclic moiety containing 1 to 4 heteroatoms selected from O, N, and S. Cycloheteroaliphatic groups optionally contain one or more double bonds. Cycloheteroaliphatic groups include, but are not limited to, azetidinyl, tetrahydrofuranyl, imidazolidinyl, pyrrolidinyl, piperidinyl, piperazinyl, oxazolidinyl, thiazolidinyl, pyrazolidinyl, thiomorpholinyl, tetrahydrothiazinyl, tetrahydro-thiadiazinyl, morpholinyl, oxetanyl, tetrahydrodiazinyl, oxazinyl, oxathiazinyl, indolinyl, isoindolinyl, quinuclidinyl, chromanyl, isochromanyl, and benzoxazinyl. Nonlimiting examples of monocyclic saturated or partially saturated ring systems are tetrahydrofuran-2-yl, tetrahydrofuran-3-yl, imidazolidin-1-yl, imidazolidin-2-yl, imidazolidin-4-yl, pyrrolidin-1-yl, pyrrolidin-2-yl, pyrrolidin-3-yl, piperidin-1-yl, piperidin-2-yl, piperidin-3-yl, piperazin-1-yl, piperazin-2-yl, piperazin-3-yl, 1,3-oxazolidin-3-yl, isothiazolidine, 1,3-thiazolidin-3-yl, 1,2-pyrazolidin-2-yl, 1,3-pyrazolidin-1-yl, thiomorpholin-yl, 1,2-tetrahydrothiazin-2-yl, 1,3-tetrahydrothiazin-3-yl, tetrahydrothiadiazin-yl, morpholin-yl, 1,2-tetrahydrodiazin-2-yl, 1,3-tetrahydrodiazin-1-yl, 1,4-oxazin-2-yl, and 1,2,5-oxathiazin-4-yl. Cycloheteroaliphatic groups may be unsubstituted or substituted by one or more suitable substituents.

The term “(hetero)aliphatic” refers to and is intended to constitute a disclosure of both aliphatic and heteroaliphatic, and it should be understood that wherever (hetero)aliphatic is referenced, it can be replaced by aliphatic and/or heteroaliphatic. Likewise, “cyclo(hetero)aliphatic” refers to and is intended to constitute a disclosure of both cycloaliphatic and cycloheteroaliphatic and can be replaced by cycloaliphatic and/or cycloheteroaliphatic wherever it appears.

“Cyclo(hetero)aliphatic-(hetero)aliphatic” refers to an acyclic (hetero)aliphatic group in which one of the hydrogen atoms bonded to a carbon atom, typically a terminal carbon atom, is replaced with a cycloaliphatic or cycloheteroaliphatic group. The term also includes a (hetero)aliphatic-cyclo(hetero)aliphatic-(hetero)aliphatic arrangement. Cycloaliphatic-(hetero)aliphatic groups may be unsubstituted or substituted by one or more suitable substituents.

The term “aromatic” refers to cyclic groups that are planar and comprise 4n+2 electrons, such as phenyl, naphthyl, anthracyl, indanyl, and the like. Aromatic groups may be unsubstituted or substituted by one or more suitable substituents. “Heteroaromatic” refers to an aromatic group in which one or more carbon atoms have been replaced with a heteroatom, such as, O, N, or S. The term “heteroaromatic” refers to a monocyclic or bicyclic 5- or 6-membered ring system, wherein the heteroaromatic group is unsaturated and satisfies Hückel's rule. Non-limiting examples of heteroaromatic groups include furanyl, thiophenyl, pyrrolyl, pyrazolyl, imidazolyl, 1,2,3-triazolyl, 1,2,4-triazolyl, isoxazolyl, oxazolyl, isothiazolyl, thiazolyl, 1,3,4-oxadiazol-2-yl, 1,2,4-oxadiazol-2-yl, 5-methyl-1,3,4-oxadiazole, 3-methyl-1,2,4-oxadiazole, pyridinyl, pyrimidinyl, pyrazinyl, triazinyl, benzofuranyl, benzothiophenyl, indolyl, quinolinyl, isoquinolinyl, benzimidazolyl, benzoxazolinyl, benzothiazolinyl, quinazolinyl, and the like. Heteroaromatic groups may be unsubstituted or substituted.

“Aromatic-(hetero)aliphatic” refers to an acyclic (hetero)aliphatic group in which one of the hydrogen atoms bonded to a carbon atom, typically a terminal carbon atom, is replaced with an aromatic group. Typical aromatic-aliphatic groups include, but are not limited to, benzyl, 2-phenylethan-1-yl, naphthylmethyl, 2-naphthylethan-1-yl, naphthobenzyl, 2-naphthophenylethan-1-yl and the like. The term also includes a (hetero)aliphatic-aromatic-(hetero)aliphatic arrangement. Aromatic-(hetero)aliphatic groups may be unsubstituted or substituted by one or more suitable substituents.

The term “carbonyl” refers to a substituent comprising a carbon double bonded to an oxygen. Examples of such substituents include aldehydes, ketones, carboxylic acids, esters, amides, carbonates, and carbamates. Carbonyl groups may be unsubstituted or substituted by one or more suitable substituents, as defined above.

The term “amino” refers to any nitrogen-containing moiety. Non-limiting examples of the amino group are NH₂— (primary), RHN— (secondary), and R₂N(tertiary) where R is alkyl, alkenyl, alkynyl, aryl, heterocyclic, or heteroaryl. RHN— and R₂N groups may be unsubstituted or substituted, as defined above.

“Halogen” or “halo” refers to fluorine, chlorine, bromine, and iodine.

The term “substituted” in reference to aliphatic, cycloaliphatic, cycloaliphatic-aliphatic, aromatic, aromatic-aliphatic, heteroaromatic, heteroaromatic-aliphatic, alkyl, heteroalkyl, alkenyl, heteroalkenyl, alkynyl, heteroalkynyl, aryl, heteroaryl, heterocycle etc., for example, “substituted alkyl”, “substituted heteroalkyl”, “substituted alkenyl”, substituted heteroalkenyl”, “substituted alkynyl”, “substituted heteroalkynyl”, “substituted aryl”, “substituted heteroaryl”, and “substituted heterocycle” means alkyl, heteroalkyl, alkenyl, heteroalkenyl, alkynyl, heteroalkynyl, aryl, heteroaryl, heterocycle, respectively, in which one or more hydrogen atoms are each independently replaced with a non-hydrogen substituent. Typical substituents include, but are not limited to, —X, —R, —O—, ═O, —OR, —SR, —S—, —NR₂, —N⁺R₃, ═NR, —CX₃, —CN, —OCN, —SCN, —N═C═O, —NCS, —NO, —NO₂, ═N₂, —N₃, —NHC(═O)R, —NHS(═O)₂R, —C(═O)R, —C(═O)NRR—S(═O)₂O—, —S(═O)₂OH, —S(═O)₂R, —OS(═O)₂OR, —S(═O)₂NR, —S(═O)R, —OP(═O)(OR)₂, —P(═O)(OR)₂, —P(═O)(O—)₂, —P(═O)(OH)₂, —P(O)(OR)(O—), —C(═O)R, —C(═O)OR, —C(═O)X, —C(S)R, —C(O)OR, —C(O)O—, —C(S)OR, —C(O)SR, —C(S)SR, —C(O)NRR, —C(S)NRR, —C(═NR)NRR, where each X is independently a halogen: F, Cl, Br, or I; and each R is independently H, alkyl, aryl, arylalkyl, a heterocycle, or a protecting group. Alkylene, alkenylene, and alkynylene groups may also be similarly substituted. When the number of carbon atoms is designated for a substituted group, the number of carbon atoms refers to the group, not the substituent (unless otherwise indicated). For example, a C₁₋₄ substituted alkyl refers to a C₁₋₄ alkyl, which can be substituted with groups having more than, e.g., 4 carbon atoms.

Whenever a range of the number of atoms in a structure is indicated (e.g., a C₁-C₂₄ alkyl, C₂-C₂₄ alkenyl, C₂-C₂₄ alkynyl, etc.), it is specifically contemplated that the substituent can be described by any of the carbon atoms in the sub-range or by any individual number of carbon atoms falling within the indicated range. By way of example, a description of the group such as an alkyl group using the recitation of a range of 1-24 carbon atoms (e.g., C₁-C₂₄), 1-6 carbon atoms (e.g., C₁-C₆), 1-4 carbon atoms (e.g., C₁-C₄), 1-3 carbon atoms (e.g., C₁-C₃), or 2-24 carbon atoms (e.g., C₂-C₂₄) encompasses and specifically describes an alkyl group having any of 1, 2, 3, 4, 5, 6, 7, 8, 9, 10, 11, 12, 13, 14, 15, 16, 17, 18, 19, 20, 21, 22, 23 and 24 carbon atoms, as appropriate, as well as any sub-range thereof (e.g., 1-2 carbon atoms, 1-3 carbon atoms, 1-4 carbon atoms, etc., including but not limited to 3-18 carbon atoms, 4-12 carbon atoms, and 6-8 carbon atoms).

### EXAMPLES

### Example 1

In this example, an embodiment of the present polyfuran monomer (polyfuran monomer A) is described, along with an embodiment of a method of making a polyfuran monomer. Isophorone diisocyanate (300 g; 2.68 eq.) and Dibutyltin dilaurate (0.42 g; 750 ppm) were introduced into a kettle equipped with a stirrer, an inert gas atmosphere, and means to control the temperature, and cooled to 10° C. To the stirred solution, furfuryl alcohol (157.8 g; 1.61 eq.) was added with cooling over 1 hour at a rate to keep the temperature between 8 and 10° C. After all the alcohol was added, the solution was warmed to 60° C. as follows: (a) allow the temperature to rise over 40 minutes to 20° C., (b) hold for 90 minutes at 20° C., (c) warm to 60° C. over 45 minutes, and (d) hold for 60 minutes at 60° C.

Tris-ethoxylated trimethylolpropane (99.14 g; 1.071 eq.) was added to the solution in three portions over fifteen minutes, controlling the temperature below 115° C. The solution was held at 110 to 115° C. for 30 minutes with vigorous stirring. A clear, light liquid was discharged and allowed to cool to a brittle solid. The resulting polyfuran monomer A had a calculated average functionality of 2.57 furans per molecule and an equivalent weight of 347 g/mole of furan.

### Example 2

In this example, an embodiment of the present polyfuran monomer (polyfuran monomer B) is described, along with an embodiment of a method of making a polyfuran monomer. Isophorone diisocyanate (359.1 g; 3.20 eq.) and Dibutyltin dilaurate (0.50 g; 750 ppm) were introduced into a kettle equipped with a stirrer, an inert gas atmosphere, and means to control the temperature, and cooled to 10° C. To the stirred solution, furfuryl alcohol (157.36 g; 1.60 eq.) was added with cooling over 1 hour at a rate to keep the temperature between 15 and 20° C. After all the alcohol was added, the solution was warmed to 60° C. as follows: (a) allow the temperature to rise over 60 minutes to 30° C., (b) warm to 60° C. over 20 minutes, and (c) hold for 60 minutes at 60° C.

Tris-ethoxylated trimethylolpropane (148.34 g; 1.60 eq.) was added to the solution in three portions over fifteen minutes, controlling the temperature below 115° C. The solution was held at 115 to 120° C. for 15 minutes with vigorous stirring. A clear, light liquid was discharged and allowed to cool to a brittle solid. The resulting polyfuran monomer B had a calculated average functionality of 3.0 furans per molecule and an equivalent weight of 415 g/mole of furan.

### Example 3

In this example, an embodiment of the present polyfuran monomer (polyfuran monomer C) is described, along with an embodiment of a method of making a polyfuran monomer. Isophorone diisocyanate (301.5 g; 2.69 eq.) and Dibutyltin dilaurate (0.40 g; 750 ppm) were introduced into a kettle equipped with a stirrer, an inert gas atmosphere, and means to control the temperature, and cooled to 10° C. To the stirred solution, furfuryl alcohol (158.56 g; 1.62 eq.) was added with cooling over 1 hour at a rate to keep the temperature between 15 and 20° C. After all the alcohol was added, the temperature was increased to 60° C. over 60 minutes. The solution was held for 30 minutes at 60° C., then warmed to 100° C.

Di-trimethylolpropane (67.36 g; 1.08 eq.) was added to the solution in three portions over fifteen minutes, controlling the temperature between 10° and 130° C. The solution was held at 125 to 130° C. for 15 minutes with vigorous stirring. A clear, light-colored liquid was discharged and allowed to cool to a brittle solid. The resulting polyfuran monomer C had a calculated average functionality of 3.0 furans per molecule and an equivalent weight of 327 g/mole of furan.

### Example 4

In this example, an embodiment of the present polyfuran/polymaleimide copolymer (polyfuran/polymaleimide Copolymer D) is described, along with an embodiment of a method of making a polyfuran/polymaleimide copolymer.

Powdered, solid polyfuran A (180.9 g; 0.52 eq.) and powdered, solid 1,6-Hexamethylenebismaleimide (72.0 g; 0.52 eq.) were combined. A molten mixture was made by heating the solid mixture with stirring to 170° C. The mixture was stirred at 170° C. for 2 minutes and cooled to room temperature over less than 10 minutes. The clear, brittle, light red solid was ground to a powder to produce Polyfuran/Polymaleimide Copolymer D.

### Example 5

In this example, an embodiment of the present polyfuran/polymaleimide copolymer (Polyfuran/Polymaleimide Copolymer E) is described, along with an embodiment of a method of making a polyfuran/polymaleimide copolymer.

Powdered, solid polyfuran B (15.00 g; 0.0361 eq.) and 1,6-Hexamethylenebismaleimide (4.99 g; 0.0361 eq.) were combined. A molten mixture was made by heating the solid mixture with stirring to 170° C. The mixture was stirred at 170° C. for 2 minutes and cooled to room temperature over less than 5 minutes. The clear, brittle, light red solid was ground to a powder to produce Polyfuran/Polymaleimide Copolymer E.

### Example 6

In this example, an embodiment of the present polyfuran/polymaleimide copolymer (Polyfuran/Polymaleimide Copolymer F) is described, along with an embodiment of a method of making a polyfuran/polymaleimide copolymer.

Powdered, solid polyfuran C (14.50 g; 0.0444 eq.) and 1,6-Hexamethylenebismaleimide (6.13 g; 0.0444 eq.) were combined. A molten mixture was made by heating the solid mixture with stirring to 175° C. The mixture was stirred at 175° C. for 2 minutes and cooled to room temperature over less than 5 minutes. The clear, brittle, light red solid was ground to a powder to produce Polyfuran/Polymaleimide Copolymer F.

### Example 7

In this example, an embodiment of the present reusable composite is described, along with an embodiment of a method of making a reusable composite. A composite lay-up was prepared using four layers of +45/−45 glass fabric (121.0 g) and five layers of powdered copolymer D (80.68 g) The lay-up was vacuum bagged against a light steel caul plate (which had been treated with a mold release agent), using a peel ply and a bleeder ply. Vacuum was established, and the vacuum bagged lay-up was transferred into an oven preheated to 170-175° C. The bagged lay-up was held in the oven under vacuum for 25 minutes, then allowed to cool over about 20 minutes to form a composite part. The composite part was then removed from the vacuum bag and cut into specimens for the Flexural Strength test (ASTM D790).

### Example 8

In this example, another embodiment of the present reusable composite is described, along with an embodiment of a method of making a reusable composite. A composite lay-up was prepared using four layers of +45/−45 glass fabric (111.5 g) and five layers of powdered copolymer E (74.3 g). The lay-up was vacuum bagged against a light steel caul plate (which has been treated with a mold release agent), using a peel ply and a bleeder ply. Vacuum was established, and the vacuum bagged lay-up was transferred into an oven preheated to 170-175° C. The bagged lay-up was held in the oven under vacuum for 25 minutes, then allowed to cool over about 20 minutes to form a composite part. The composite part was then removed from the vacuum bag and was cut into specimens for the Flexural Strength test (ASTM D790).

### Example 9

In this example, another embodiment of the present reusable composite is described, along with an embodiment of a method of making a reusable composite. A composite lay-up was prepared using four layers of +45/−45 glass fabric (100.68 g) and five layers of powdered copolymer F (67.12 g). The lay-up was vacuum bagged against a light steel caul plate (which has been treated with a mold release agent), using a peel ply and a bleeder ply. Vacuum was established, and the vacuum bagged lay-up was transferred into an oven preheated to 175-180° C. The bagged lay-up was held in the oven under vacuum for 25 minutes, then transferred still under a vacuum to a 100° C. and held it for 30 minutes. The vacuum was broken, and the material was annealed by cooling to 70° C. at the following schedule: (a) 15 minutes at 90° C.; (b) 15 minutes at 80° C.; (c) 40 minutes at 70° C., thereby producing a composite part.

The composite part removed from the vacuum bag was well compacted and hard but exhibited surface pattern that had transferred from the bleeder ply. To repair this defect, the composite part was reprocessed as follows. It was heated to 180° C. between polished steel plates that had been previously treated with mold release. A pressure of 25 psi was applied. The composite part was held 30 minutes at 180° C. and cooled to 120° C. over 5 minutes (12° C./minutes). The pressure was released, and the composite part was annealed by cooling to 70° C. over 120 minutes (0.42° C./minute). The surface profile of the final composite part was now smooth.

### Example 10

In this example, half of the composite part specimens made in Example 8 were annealed as follows: (1) Heat to 80° C. and hold for 60 minutes; (2) Cool to 70° C. and hold for 60 minutes; (3) Allow to cool to room temperature over about 5 minutes.

### Example 11

In this example, the self-repair properties of the present composite parts were evaluated. Several broken composite articles prepared in Example 7 were repaired by the following process.

After having been tested using ASTM D790, the broken composite articles were vacuum bagged against a steel caul plate that had been previously treated with a mold release agent. Vacuum was established, and the vacuum bagged parts were transferred into an oven preheated to 170-175° C. The bagged lay-up was held in the oven under vacuum for 20 minutes. The bagged parts were annealed as follows: (a) Transfer the bagged parts to a 90° C. oven and hold for 30 minutes; (b) Cool oven to 80° C. and hold for 60 minutes; (c) Cool oven to 70° C. and hold for 60 minutes. The parts were allowed to cool to room temperature over about 5 minutes.

### Analysis of Mechanical Testing Results

Specimens prepared in Examples 7, 8, 10 and 11 were tested for flexural strength using ASTM D790. The results are shown in Table 1.

TABLE 1
Flexural Strength of Composite Parts

| | | Strength | Strain | Modulus |
|---|---|---|---|---|
| Example | Notes | (psi) | (%) | (ksi) |
| 7 | Furan Functionality 2.6 | 7,220 | 1.40 | 11.3 |
| 8 | Furan Functionality 3.0 | 11,760 | 1.76 | 17.4 |
| 10 | Furan Functionality 3.0, | 14,150 | 2.15 | 15.0 |
| | Annealed | | | |
| 11 | Broken Specimens from Exp. | 11,530 | 1.52 | 16.3 |
| | 8 Healed and Annealed | | | |

These results demonstrate that (1) the properties of the present composites may be tuned by adjusting polyfuran resin selection; (2) the properties of the present composites may be improved by annealing; and (3) Broken or malformed composite parts may be repaired by reheating, reforming and annealing.

Separation of Composite Parts into Fiber and Resin Fractions

Examples 12 to 14 demonstrate that the concept of dissolving polyfuran/polymaleimide Diels-Alder adducts by heating in a high-boiling solvent is known. We ran several experiments to explore the process of removing our polymer from a glass composite by this method.

### Example 12

This example demonstrates an embodiment of a method of reclaiming the present reusable matrix-forming resin from a fiberglass composite by separating the resin from glass fibers. The resin was removed from a fiber/resin article by the following process:
(1) A solid panel about 8×8×⅛ inches was prepared from fiberglass (106.53 g) and Copolymer E (44.27 g) using the process outlined in Example 9 above.
(2) The panel was cut into pieces approximately ¾ by 4 inches. This process removed a portion of the composite part as sawdust and contaminated scrap. The cut pieces were placed in a stirred vessel under nitrogen atmosphere with 2-heptanone (100 g) and mixed mesitylenes (200 g).
(3) The vessel was raised to 155° C., whereupon the resin dissolved, leaving loose fiber.
(4) The mixture was filtered to remove the resin solution, rinsed with additional hot 2-heptanone, then with hexane. After drying, 94.11 g of loose fiber was recovered.
(5) The 2-heptanone/mesitylene solution was cooled at 10° C., resulting in separation of yellow oil. After rinsing with additional hexane and drying for a period of 2 weeks a mixture of solid, gummy solid and oil was recovered, about 40 grams.

### Example 13

This example demonstrates an embodiment of a method of reclaiming the present reusable matrix-forming resin by separating the resin from glass fibers.
(1) Copolymer D (14.69 g) was dissolved in dry dimethylformamide (DMF) (103 g) by heating to 152° C. in a stirred vessel under nitrogen atmosphere.
(2) This solution was cooled over less than five minutes to 100° C. At 100° C. the dissolved polymer, which would include a fraction of depolymerized polyfuran and polymaleimide Diels-Alder precursors substantially repolymerizes. To maximize re-polymerization, the solution was then annealed by cooling from 100° C. to 50° C. over 3 hours. Considered completely repolymerized, it was allowed to cool to 20° C. over less than five minutes.
(3) The solution was poured with stirring into a volume of deionized (D.I.) water (500 g) over about 2 minutes, precipitating an off-white particulate solid.
(4) The water-DMF/particulate solid mixture was filtered and the solid washed with 100 g D.I. water.
(5) The particulate solid was re-slurried into water (200 g) and stirred for five minutes, then filtered and washed with D.I. water (100 g).
(6) Step 5 was repeated.
(7) The washed particulate solid was dried overnight at 45° C. in an air-circulating oven to a constant weight of 13.82 g, a recovery of 94.1%. This powdered solid was assigned the name Reclaimed Copolymer D.

The infrared spectrum of the recovered Reclaimed Copolymer D. from step 7 was found to be essentially identical to the spectrum from the starting Copolymer D.

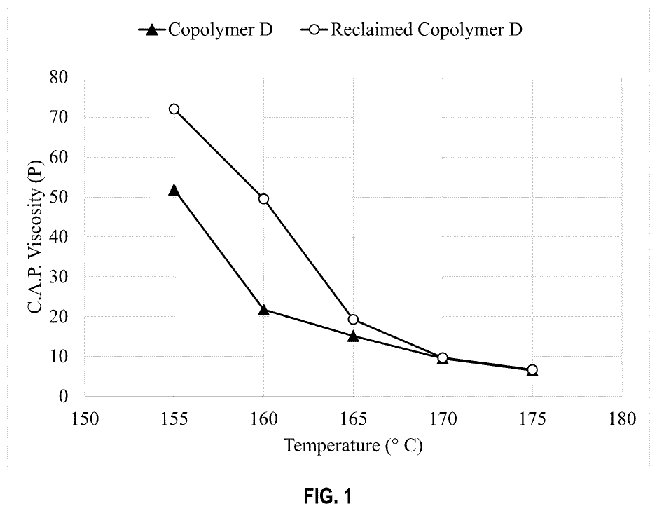

To be useful as a matrix resin a polymer must have acceptable viscosity at a convenient processing temperature. Viscosities were determined using a cone and plate type viscometer, Brookfield Model 2000+. This viscometer operates by holding a liquid polymer sample between a flat plate and a conical spindle such that the surface of the spindle is immersed in the polymer, holding the plate, polymer and spindle at a pre-set temperature, rotating the spindle at a pre-set rate while holding the plate stationary, and measuring the resistance to turning. FIG. 1 compares the viscosities of Copolymer D and Reclaimed Copolymer D, which shows that they have similar viscosities at typical processing temperature of 165-175° C.

The increased viscosity from Copolymer D to Reclaimed Copolymer D might be explained by the coincident loss of 6% of the polymer mass that occurred during the reclamation process. This lost component may have included low molecular weight species soluble in the DMF/water blend which could act as plasticizers.

### Example 14

This example demonstrates a closed loop recycling process with an embodiment of the present reusable matrix-forming resin.

As Example 14.1, a batch of Copolymer D was prepared by the method of Example 3, combining Polyfuran Monomer A (112.00 g), and 1,6-Hexamethylenebismaleimide (44.56 g). The yield of ground and sieved product was 152.48 g (97.4%).

As Example 14.2, two composite panels 14A and 14B were prepared by the method of Example 7 using a portion of the Copolymer D prepared in Example 14.1. Four sheets of PGTex Biax 806 and five layers of the powdered polyfuran/polymaleimide copolymer D from Ex. 14.1 were combined in on each part. In a modification of the process from Example 7, the formed panels were annealed by cooling from 100 to 70° C. at 0.125° C./minute.

Panel 14A weighed 156.25 g and contained 113.76 g (72.8%) glass and 42.49 g (27.2% resin). Panel 14B weighed 150.77 g and contained 114.45 g (75.9%) glass and 36.32 g (24.1.2% resin).

Various composite parts containing Copolymer D were cut into approximately 1×1-inch squares. These parts had an overall composition as presented in Table 2.

TABLE 2
Composition of Composite Parts Before Recycling

| | | | Approximate |
|---|---|---|---|
| | | Approximate Glass | Copolymer D |
| Part | Mass (g) | Content (g) | Content (g) |
| 14A | 130.8 | 95.5 | 35.3 |
| Bleeder Plies from | 92.0 | 35 | 57 |
| 14A and 14B | | | |
| Total | 223 | 131 | 92 |

Table 2 includes 'Bleeder Plies from 14A and 14B. A layup for producing composite parts in a vacuum bag may include a “bleeder ply,” which is generally a loose fiber layer, either woven or non-woven, whose function is to absorb excess resin released when the desired part is compacted under the pressure produced by the vacuum bag. The bleeder ply is typically separated from the composite part by a lightly perforated peel ply, a non-bonding layer which allows the part to be separated from the bagging materials. The resin was extracted as follows from the sectioned parts and sectioned bleeder plies by the following process (an embodiment similar to the process of Example 13, but starting with composite articles rather than neat resin). (a) Combine the composite articles (223 g, containing approximately 92 g of resin), with dry DMF (616 g) in a 2-liter kettle under inert gas atmosphere. (b) Heat to about 152° C. over about 30 minutes. This dissolved the resin and released the fibers. (c) Remove the heater and allow the kettle to cool to 120° C. over about 5 minutes. (d) Insulate the kettle so that it cooled at a rate of about 0.33° C./min for several hours. (e) Pour the DMF solution of the resin out of the mat of loose glass fibers into 4 liters of deionized water at about 18° C. to precipitate an off-white solid. Rinse the fiber mat with an additional 100 g of DMF and add the DMF+ to the water solution. (f) Filter the solid polymer from the DMF/water solution. (g) Re-slurry the filtered solid in 700 g water. Stir for 15 minutes then filter. Wash filter with 100 g D.I. water. (h) Repeat re-slurry and rinse. (i) The rinsed particulate solid was dried overnight at 55° C. in an air-circulating oven to a constant weight of 75.75 g, a recovery of 82%. This powdered solid was assigned the name Reclaimed Copolymer D. (j) Rinse the reclaimed fiber mat with 800 ml of acetone. Filter and allow the fiber mat to dry to 146.0 g, a recovery of 111%. A patchy distribution of resin retained on the glass suggested limitations of this small-scale laboratory extraction experiment rather than a broad, uniform failure of a portion of the resin to dissolve.

Using the process of Example 14.2 (comprising combining powdered resin, and fiber plies to make articles, followed by annealing of the articles), four sheets of PGTex Biax 806 fiberglass were combined with five layers of Reclaimed Copolymer D to prepare Panel 14C. This panel contained 110.04 g (70.0%) glass fiber and 47.05 g (30.0%) resin.

The composite panels produced from virgin Copolymer D in Example 14. 2 above (Panel 14B) and Panel 14C from Reclaimed Copolymer D were sectioned and tested for flexural strength, strain and modulus using the procedure of ASTM D790. Results are compared below in Table 3. Also included are the results from Example 7 wherein virgin Copolymer D was used, but the resulting part was not annealed.

TABLE 3
Flexural Strength of Composite Parts

| | | Strength | Strain | Modulus |
|---|---|---|---|---|
| Example | Source | (psi) | (%) | (ksi) |
| 7 | From Virgin Copolymer D, | 7,220 | 1.40 | 11.3 |
| | Not Annealed | | | |
| 14.2 | Panel 14B, from Virgin | 11,907 | 1.54 | 12.5 |
| | Copolymer D, Annealed | | | |
| 14.5 | Panel 14C, from | 12,284 | 1.37 | 11.7 |
| | Reclaimed Copolymer D, | | | |
| | Annealed | | | |

Overall, Example 14 demonstrates that the inventive Diels-Alder matrix copolymers may be reclaimed from end-of-life composite parts and recycled as matrix-forming resins to form matrix copolymers in new composite articles.

1. A polyfuran monomer having a structure of Formula I, II or III: 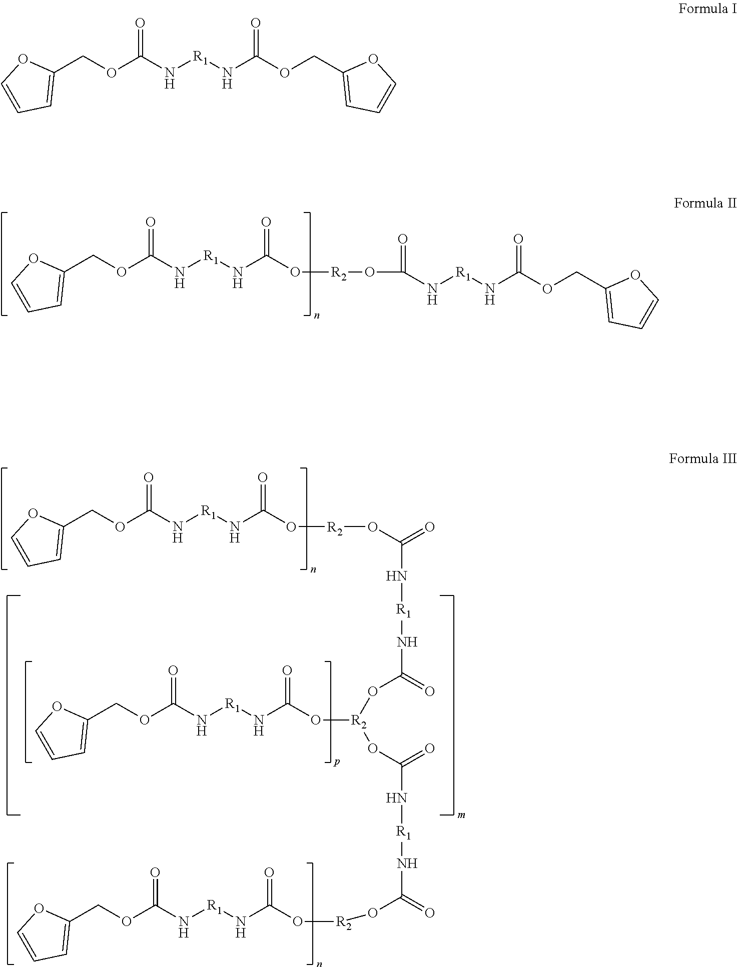
wherein R¹ is an aliphatic, heteroaliphatic, cycloaliphatic, cycloheteroaliphatic, cyclo(hetero)aliphatic-(hetero)aliphatic, aromatic, aromatic-(hetero)aliphatic, heteroaromatic or heteroaromatic-(hetero)aliphatic group, all of which are optionally substituted;
wherein R² is an aliphatic, cycloaliphatic, cycloaliphatic-aliphatic, aromatic, aromatic-aliphatic, heteroaromatic or heteroaromatic-aliphatic group, all of which are optionally substituted;
m is 0 or more;
n is 1 or more; and
p is 1 or more.

2. The polyfuran monomer of claim 1, wherein the polyfuran monomer has at least three furan functionalities per molecule.

3. The polyfuran monomer of claim 1, wherein the polyfuran monomer has an average of at least 2.25 furan functionalities per molecule.

4. The polyfuran monomer of claim 1, wherein R¹ is an optionally substituted cycloaliphatic group, an optionally substituted cycloaliphatic-aliphatic group, an optionally substituted aromatic group, or an optionally substituted aromatic-aliphatic group.

5. The polyfuran monomer of claim 1, wherein R¹ is an optionally substituted cycloaliphatic group or an optionally substituted aromatic group.

6. The polyfuran monomer of claim 1, wherein R¹ comprises a group of Formula IVa or IVb:

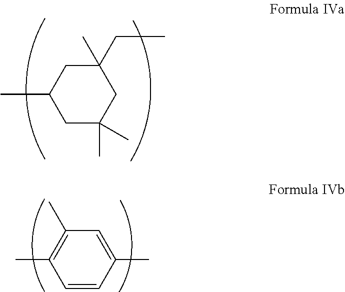

7. The polyfuran monomer of claim 1, wherein the polyfuran monomer has a structure of Formula II.

8. The polyfuran monomer of claim 1, wherein the polyfuran monomer has a structure of Formula III.

9. A reusable resin comprising:
one or more polyfuran monomers having at least two furan functionalities per molecule, wherein at least one of the polyfuran monomers is a polyfuran monomer according to claim 1; and
one or more polymaleimide monomers having at least two maleimide functionalities per molecule.

10. A polymerizable composition comprising the reusable resin of claim 9 and one or more composition components.

11. The polymerizable composition of claim 10, wherein the one or more composition components are selected from the group consisting of non-fibrous fillers, pigments, thinners, wetting agents, surfactants, flow promoting agents, leveling agents, degassing agents, antifoam agents, texturizing agents, thickeners, rheology modifiers, antioxidants, heat stabilizers, ultraviolet light stabilizers, free-radical scavengers, color-stabilizing agents, antimicrobial agents, preservatives, bacteriocides, or fungicides, and mixtures thereof.

12. A polymer article comprising the reusable resin of claim 9.

13. A reusable matrix-forming resin comprising:
one or more polyfuran monomers having at least two furan functionalities per molecule, wherein at least one of the polyfuran monomers is a polyfuran monomer according to claim 1; and
one or more polymaleimide monomers having at least two maleimide functionalities per molecule.

14. A composite composition comprising:
the reusable matrix-forming resin of claim 13, and
a reinforcing material.

15-19. (canceled)

20. A composite article comprising the composite composition of claim 14.

21. A method of making a polyfuran monomer comprising:
reacting a furfuryl alcohol with a polyisocyanate to form a polyfuran monomer having at least two furan functionalities per polyfuran monomer.

22. The method of claim 21, wherein the polyfuran monomer is made in the absence of an acid.

23. The method of claim 21, wherein the furfuryl alcohol is a hydroxyalkylfuran.

24-29. (canceled)

30. The method of claim 21, wherein the furfuryl alcohol and the polyisocyanate are reacted in a vessel, and the method further comprises adding a polymaleimide to the polyfuran monomer in the same vessel.

31. A method of forming a composite article comprising:
heating a polyfuran/polymaleimide copolymer to form a liquid resin or a melt resin;
applying the liquid resin or the melt resin to a fibrous reinforcing material to form a malleable composite material;
shaping the malleable composite material;
solidifying the shaped composite material by cooling to a solidifying temperature; and
annealing the shaped composite material.

32-48. (canceled)
